(19) United States
(12) Patent Application Publication
(10) Pub. No.: US 2024/0346648 A1
(43) Pub. Date: Oct. 17, 2024

(54) METHOD AND APPARATUS FOR CALIBRATING BLOOD FLOW VELOCITY

(71) Applicant: Medipixel, Inc., Seoul (KR)

(72) Inventors: Yeong Jun Kim, Seoul (KR); Jong Hoe Ku, Seoul (KR); Si-Hyuck Kang, Seoul (KR)

(21) Appl. No.: 18/633,668

(22) Filed: Apr. 12, 2024

(30) Foreign Application Priority Data
Apr. 14, 2023 (KR) 10-2023-0049665

(51) Int. Cl. G06T 7/00; A61B 5/0245

(52) U.S. Cl. CPC G06T 7/0012; A61B 5/0245; G06T 2207/30104

(57) ABSTRACT

A method for correcting blood flow velocity is provided, including receiving a plurality of medical images depicting a target blood vessel, receiving electrocardiogram data of an object of measurement, calculating blood flow velocity of the target blood vessel using the plurality of medical images, and correcting the calculated blood flow velocity based on the electrocardiogram data and standard blood flow velocity data of the target blood vessel.

### CROSS-REFERENCE TO RELATED APPLICATION

This application claims priority under 35 U.S.C § 119 to Korean Patent Application No. 10-2023-0049665, filed in the Korean Intellectual Property Office on Apr. 14, 2023, the entire contents of which are hereby incorporated by reference.

### BACKGROUND

### Technical Field

The present disclosure relates to a method and an apparatus for correcting blood flow velocity, and specifically, to a method and an apparatus for correcting blood flow velocity of a target blood vessel of an object of measurement based on electrocardiogram data and standard blood flow velocity data of the target blood vessel of the object of measurement.

### Description of the Related Art

The blood flow velocity acts as an important factor in calculating pressure loss, so accurately measuring the blood flow velocity is important for accurately diagnosing lesions and establishing treatment plans. In recent years, medical imaging technologies such as ultrasound Doppler, magnetic resonance imaging (MRI), and computed tomography (CT) are used in medical sites to directly measure or estimate blood flow velocities.

However, according to the related technologies, there is a problem in that the blood flow velocity is not measured at the same time point or period in the heart rate cycle of an object of measurement every time it is measured. That is, according to the related technologies, there is a problem in that it is difficult to measure the blood flow velocity independently from the influence of heart rate cycle on the blood flow velocity, and that the blood flow velocity can be measured lower or higher than the general velocity depending on a specific phase of the heart rate cycle.

### SUMMARY

In order to address one or more problems (e.g., the problems described above and/or other problems not explicitly described herein), the present disclosure provides a method, a non-transitory computer-readable recording medium, and an apparatus (system) for correcting blood flow velocity.

The present disclosure may be implemented in various ways including a method, a system (apparatus), a computer program stored in a readable storage medium or a computer-readable recording medium.

A method for correcting blood flow velocity is provided, which may be performed by one or more processors and include receiving a plurality of medical images depicting a target blood vessel, receiving electrocardiogram data of a object of measurement, calculating blood flow velocity of the target blood vessel using the plurality of medical images, and correcting the calculated blood flow velocity based on the electrocardiogram data and standard blood flow velocity data of the target blood vessel.

The correcting the calculated blood flow velocity may include determining a correction factor for correcting the blood flow velocity of the target blood vessel, and applying the determined correction factor to the blood flow velocity of the target blood vessel so as to correct the blood flow velocity.

The plurality of medical images may include a first medical image and a second medical image, and the determining the correction factor may include calculating, from the standard blood flow velocity data, a first average velocity, which is an average value per heart rate cycle of the object of measurement, of the standard blood flow velocity of the target blood vessel, calculating a second average velocity, which is an average value of the standard blood flow velocity of the target blood vessel between time when the first medical image is captured and time when the second medical image is captured, and determining the correction factor using the calculated first average velocity and the calculated second average velocity.

The method may further include mapping the standard blood flow velocity data to the electrocardiogram data such that a cycle of the standard blood flow velocity data matches a heart rate cycle of the electrocardiogram data.

The plurality of medical images may include a first medical image and a second medical image, and the calculating the blood flow velocity of the target blood vessel may include determining a distance between a first point in the target blood vessel where a contrast agent reaches in the first medical image and a second point in the target blood vessel where the contrast agent reaches in the second medical image, determining a time interval between time when the first medical image is captured and time when the second medical image is captured, and calculating blood flow velocity of the target blood vessel using the determined distance and the determined time interval.

The time interval may be determined based on, within the plurality of medical images, a number of frames between the first and second medical images and a number of frames per second of the plurality of medical images.

The standard blood flow velocity data may be data that is standardized based on at least one of age, gender, race, height, weight, presence or absence of cardiovascular disease, obesity (BMI), blood pressure, or smoking status of the object of measurement.

The target blood vessel may be one of right coronary artery (RCA), left anterior descending artery (LAD), or left circumflex artery (LCX).

There may be provided a non-transitory computer-readable recording medium storing instructions that cause performance of the method for correcting blood flow velocity on a computer.

A system for correcting blood flow velocity may be provided, which may include a communication module, a memory, and one or more processors connected to the memory and configured to execute one or more computer-readable programs included in the memory, in which the communication module may be configured to receive a plurality of medical images depicting a target blood vessel, and receive electrocardiogram data of an object of measurement, and the one or more programs may include instructions for calculating blood flow velocity of the target blood vessel using the plurality of medical images, and correcting the calculated blood flow velocity based on the electrocardiogram data and standard blood flow velocity data of the target blood vessel.

The correcting the calculated blood flow velocity may include determining a correction factor for correcting the blood flow velocity of the target blood vessel, and applying the determined correction factor to the blood flow velocity of the target blood vessel so as to correct the blood flow velocity.

The plurality of medical images may include a first medical image and a second medical image, and the determining the correction factor may include calculating, from the standard blood flow velocity data, a first average velocity, which is an average value per heart rate cycle of the object of measurement, of the standard blood flow velocity of the target blood vessel, calculating a second average velocity, which is an average value of the standard blood flow velocity of the target blood vessel between time when the first medical image is captured and time when the second medical image is captured, and determining the correction factor using the calculated first average velocity and the calculated second average velocity.

The one or more programs may further include instructions for mapping the standard blood flow velocity data to the electrocardiogram data such that a cycle of the standard blood flow velocity data matches a heart rate cycle of the electrocardiogram data.

The plurality of medical images may include a first medical image and a second medical image, and the calculating the blood flow velocity of the target blood vessel may include determining a distance between a first point in the target blood vessel where a contrast agent reaches in the first medical image and a second point in the target blood vessel where the contrast agent reaches in the second medical image, determining a time interval between time when the first medical image is captured and time when the second medical image is captured, and calculating blood flow velocity of the target blood vessel using the determined distance and the determined time interval.

According to some examples of the disclosure, by mapping the standard blood flow velocity data to the electrocardiogram data such that the cycle of the standard blood flow velocity data matches the heart rate cycle of the electrocardiogram data, it is possible to infer the general blood flow velocity or velocity trend of the target blood vessel according to positions in the electrocardiogram cycle of the object of measurement.

According to some examples of the disclosure, by excluding or minimizing the influence of the heart rate cycle of the object of measurement on the blood flow velocity of the target blood vessel of the object of measurement, the blood flow velocity data of the blood vessel of the object of measurement can be obtained.

According to some aspects of the disclosure, by providing the standardized blood flow velocity data according to the characteristics of the object of measurement, it is possible to correct the blood flow velocity more accurately.

The effects of the present disclosure are not limited to the effects described above, and other effects not mentioned will be able to be clearly understood by those of ordinary skill in the art (referred to as “those skilled in the art”) from the description of the claims.

### BRIEF DESCRIPTION OF THE DRAWINGS

The above and other objects, features and advantages of the present disclosure will be described with reference to the accompanying drawings described below, where similar reference numerals indicate similar elements, but not limited thereto, in which:

FIG. 1 is a diagram illustrating a process of calculating a corrected blood flow velocity;

FIG. 2 is a block diagram illustrating an internal configuration of a system for correcting blood flow velocity;

FIG. 3 is a block diagram illustrating an internal configuration of a processor of the system for correcting blood flow velocity;

FIG. 4 is a diagram illustrating a plurality of medical images for calculating blood flow velocity of a target blood vessel;

FIG. 5 is a diagram illustrating a graph including standard blood flow velocity data and electrocardiogram data of the target blood vessel;

FIG. 6 is a flowchart illustrating part of a process of a method for calculating and correcting blood flow velocity;

FIG. 7 is a flowchart illustrating a method for calculating blood flow velocity; and

FIG. 8 is a flowchart illustrating a method for correcting blood flow velocity.

### DETAILED DESCRIPTION

Hereinafter, example details for the practice of the present disclosure will be described in detail with reference to the accompanying drawings. However, in the following description, detailed descriptions of well-known functions or configurations will be omitted if it may make the subject matter of the present disclosure rather unclear.

In the accompanying drawings, the same or corresponding components are assigned the same reference numerals. In addition, in the following description of various examples, duplicate descriptions of the same or corresponding components may be omitted. However, even if descriptions of components are omitted, it is not intended that such components are not included in any example.

Advantages and features of the disclosed examples and methods of accomplishing the same will be apparent by referring to examples described below in connection with the accompanying drawings. However, the present disclosure is not limited to the examples disclosed below, and may be implemented in various forms different from each other, and the examples are merely provided to make the present disclosure complete, and to fully disclose the scope of the disclosure to those skilled in the art to which the present disclosure pertains.

The terms used herein will be briefly described prior to describing the disclosed example(s) in detail. The terms used herein have been selected as general terms which are widely used at present in consideration of the functions of the present disclosure, and this may be altered according to the intent of an operator skilled in the art, related practice, or introduction of new technology. In addition, in specific cases, certain terms may be arbitrarily selected by the applicant, and the meaning of the terms will be described in detail in a corresponding description of the example(s). Therefore, the terms used in the present disclosure should be defined based on the meaning of the terms and the overall content of the present disclosure rather than a simple name of each of the terms.

The singular forms “a,” “an,” and “the” as used herein are intended to include the plural forms as well, unless the context clearly indicates the singular forms. Further, the plural forms are intended to include the singular forms as well, unless the context clearly indicates the plural forms. Further, throughout the description, when a portion is stated as “comprising (including)” a component, it is intended as meaning that the portion may additionally comprise (or include or have) another component, rather than excluding the same, unless specified to the contrary.

Further, the term “module” or “unit” used herein refers to a software or hardware component, and “module” or “unit” performs certain roles. However, the meaning of the “module” or “unit” is not limited to software or hardware. The “module” or “unit” may be configured to be in an addressable storage medium or configured to play one or more processors. Accordingly, as an example, the “module” or “unit” may include components such as software components, object-oriented software components, class components, and task components, and at least one of processes, functions, attributes, procedures, subroutines, program code segments, drivers, firmware, micro-codes, circuits, data, database, data structures, tables, arrays, and variables. Furthermore, functions provided in the components and the “modules” or “units” may be combined into a smaller number of components and “modules” or “units”, or further divided into additional components and “modules” or “units.”

The “module” or “unit” may be implemented as a processor and a memory. “processor” should be interpreted broadly to encompass a general-purpose processor, a Central Processing Unit (CPU), a microprocessor, a Digital Signal Processor (DSP), a controller, a microcontroller, a state machine, and so forth. Under some circumstances, the “processor” may refer to an application-specific integrated circuit (ASIC), a programmable logic device (PLD), a field-programmable gate array (FPGA), and so on. The “processor” may refer to a combination for processing devices, e.g., a combination of a DSP and a microprocessor, a combination of a plurality of microprocessors, a combination of one or more microprocessors in conjunction with a DSP core, or any other combination of such configurations. In addition, the “memory” should be interpreted broadly to encompass any electronic component that is capable of storing electronic information. The “memory” may refer to various types of processor-readable media such as random access memory (RAM), read-only memory (ROM), non-volatile random access memory (NVRAM), programmable read-only memory (PROM), erasable programmable read-only memory (EPROM), electrically erasable PROM (EEPROM), flash memory, magnetic or optical data storage, registers, and so on. The memory is said to be in electronic communication with a processor if the processor can read information from and/or write information to the memory. The memory integrated with the processor is in electronic communication with the processor.

In the present disclosure, “target blood vessel” may refer to a blood vessel subjected to measurement and/or correction of the blood flow velocity of an object of measurement.

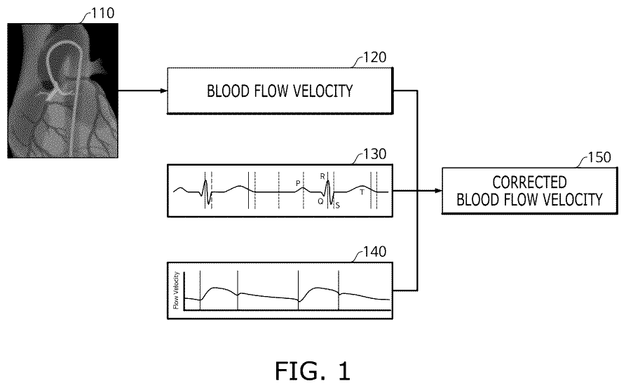

FIG. 1 is a diagram illustrating a process of calculating a corrected blood flow velocity 150. The corrected blood flow velocity 150 may be determined by correcting a blood flow velocity 120 calculated from a medical image 110 based on electrocardiogram data 130 and standard blood flow velocity data 140 of the target blood vessel.

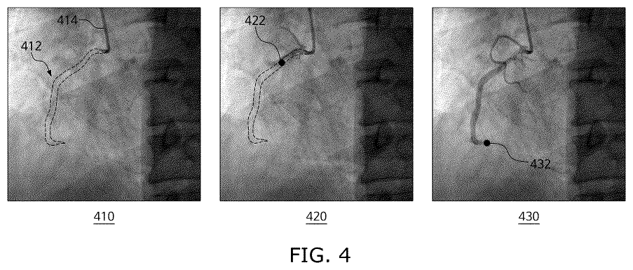

The medical image 110 may correspond to an image depicting, through X-rays or the like, a target blood vessel of an object of measurement injected with a contrast agent. The target blood vessel may correspond to one of the right coronary artery (RCA), left anterior descending artery (LAD), or left circumplex artery (LCX), but aspects are not limited thereto, and may include blood vessels such as cerebrovascular, pulmonary blood vessels, renal arteries, abdominal arteries, iliac arteries. FIG. 1 illustrates that the medical image 110 is a single image, but aspects are not limited thereto, and the medical image 110 may include a plurality of images. The process of calculating the blood flow velocity 120 from the medical image 110 will be described below in detail with respect to FIG. 4.

The standard blood flow velocity data 140 of the target blood vessel may be a standardized value calculated from pre-measured blood flow velocity data of certain subjects. The certain subjects as used herein may include subjects different from the object of measurement. Additionally or alternatively, the certain subjects may include the object of measurement. For example, the standard blood flow velocity data 140 of the target blood vessel may be determined from the blood flow velocity of the target blood vessel of each of a plurality of certain subjects.

The standard blood flow velocity data 140 may correspond to data that is standardized based on at least one of age, gender, race, height, weight, presence or absence of cardiovascular disease, obesity (BMI), blood pressure, or smoking status of the certain subjects. The standard blood flow velocity data 140 may correspond to data that is selected from a blood flow velocity data set based on information on the object of measurement. That is, data from the blood flow velocity data set, which has the largest amount of meta information matching the information on the object of measurement, may be selected as the standard blood flow velocity data 140. With this configuration, the standardized blood flow velocity data may be provided according to the characteristics of the object of measurement, thereby more accurately correcting the blood flow velocity.

The standard blood flow velocity data 140 may correspond to data that stores blood flow velocity values at each of a plurality of positions on a cycle (e.g., ¼ cycle, ½ cycle, ¾ cycle, etc.), or to data according to a blood flow velocity function. The standard blood flow velocity data 140 may be mapped to the electrocardiogram data 130 such that the cycle of the standard blood flow velocity data 140 matches the heart rate cycle of the electrocardiogram data 130. With this configuration, a general blood flow velocity or velocity trend of the target blood vessel according to the position on the electrocardiogram cycle of the object of measurement may be inferred.

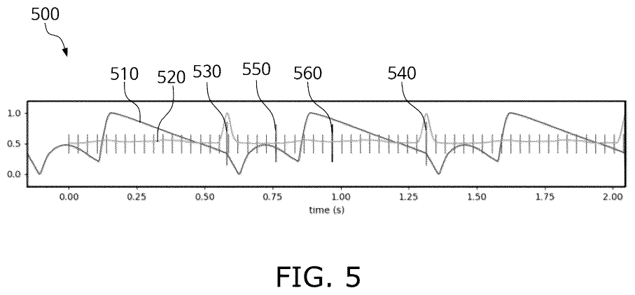

The blood flow velocity 120 may be corrected based on the electrocardiogram data 130 of the object of measurement and the standard blood flow velocity data 140 of the target blood vessel. Specifically, after a correction factor for correcting the blood flow velocity of the target blood vessel of the object of measurement is determined, the determined correction factor may be applied to the blood flow velocity 120 of the target blood vessel, thereby correcting the blood flow velocity 120. The method for correcting blood flow velocity will be described below in detail with reference to FIG. 5.

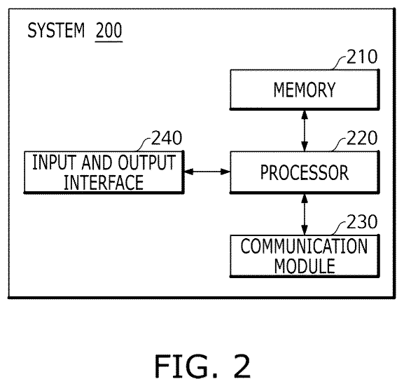

FIG. 2 is a block diagram illustrating an internal configuration of a system 200 for correcting blood flow velocity. The system 200 for correcting blood flow velocity may include a memory 210, a processor 220, a communication module 230, and an input and output interface 240. As illustrated in FIG. 2, the system 200 for correcting blood flow velocity may be configured to communicate information and/or data through a network by using the communication module 230. The system 200 for correcting blood flow velocity may be configured to communicate information and/or data with an external database (e.g., a database in which blood flow velocity data set is stored, etc.) through a network using the communication module 230.

The memory 210 may include any non-transitory computer-readable recording medium. The memory 210 may include a permanent mass storage device such as read only memory (ROM), disk drive, solid state drive (SSD), flash memory. As another example, a non-perishable mass storage device such as a ROM, SSD, flash memory, disk drive, etc. may be included in the system 200 for correcting blood flow velocity as a separate permanent storage device separate from the memory. In addition, an operating system and at least one program code may be stored in the memory 210.

These software components may be loaded from a computer-readable recording medium separate from the memory 210. This separate computer-readable recording medium may include a recording medium directly connectable to the system 200 for correcting blood flow velocity, and may include, for example, a computer-readable recording medium such as a floppy drive, a disk, a tape, a DVD/CD-ROM drive, a memory card, etc. In another example, the software components may be loaded into the memory 210 through the communication module 230 rather than the computer-readable recording medium. For example, at least one program may be loaded into the memory 210 based on a computer program installed by files provided by developers or a file distribution system that distributes an installation file of an application through the communication module 230.

The processor 220 may be configured to process the commands of the computer program by performing basic arithmetic, logic, and input and output operations. The commands may be provided to a user terminal (not illustrated) or another external system by the memory 210 or the communication module 230. In addition, the processor 220 may be configured to manage, process, and/or store the information and/or data received from a plurality of user terminals and/or a plurality of external systems.

The communication module 230 may provide a configuration or function for the user terminal (not illustrated) and the system 200 for correcting blood flow velocity to communicate with each other through a network, and may provide a configuration or function for the system 200 for correcting blood flow velocity to communicate with an external system (e.g., with a standard blood flow velocity database, etc.). For example, control signals, instructions, data, etc. provided under the control of the processor 220 of the system 200 for correcting blood flow velocity may be transmitted to the user terminal and/or the external system through the communication module 230 and the network through the communication module of the user terminal and/or the external system.

In addition, the input and output interface 240 of the system 200 for correcting blood flow velocity may be a means for interfacing with an apparatus (not illustrated) for inputting or outputting, which may be connected to, or included in the system 200 for correcting blood flow velocity. In FIG. 2, the input and output interface 240 is illustrated as a component configured separately from the processor 220, but aspects are not limited thereto, and the input and output interface 240 may be configured to be included in the processor 220. The system 200 for correcting blood flow velocity may include more components than those illustrated in FIG. 2. Meanwhile, most of the related components may not necessarily require exact illustration.

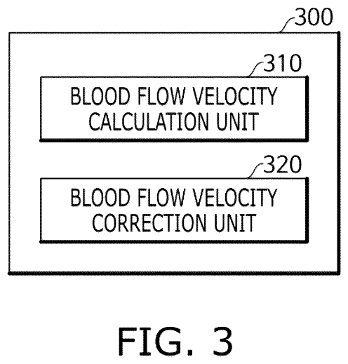

FIG. 3 is a block diagram illustrating an internal configuration of a processor 300 of the system for correcting blood flow velocity. The processor 300 may correspond to a processor included in the system for correcting blood flow velocity. As illustrated, the processor 300 may include a blood flow velocity calculation unit 310 and a blood flow velocity correction unit 320, but aspects are not limited thereto.

The blood flow velocity calculation unit 310 may use a plurality of medical images to calculate a blood flow velocity of the target blood vessel of the object of measurement. In order to calculate the blood flow velocity of the target blood vessel of the object of measurement, the blood flow velocity calculation unit 310 may determine a distance between a first point in the target blood vessel where the contrast agent reaches in the first medical image and a second point in the target blood vessel where the contrast agent reaches in the second medical image.

In addition, the blood flow velocity calculation unit 310 may determine a time interval between time when the first medical image is captured and time when the second medical image is captured. In this case, the blood flow velocity calculation unit 310 may determine the time interval based on the number of frames between the first and second medical images and the number of frames per second of the plurality of medical images in the plurality of medical images. For example, if there are two frames between the first and the second medical images and a plurality of medical images are captured at 12 frames per second, because the second medical image is at least 3 frames apart from the first medical image, the time interval may be determined to be 0.25 seconds.

The blood flow velocity calculation unit 310 may calculate the blood flow velocity of the target blood vessel using the determined distance and the determined time interval. The blood flow velocity calculation unit 310 may divide the determined distance by the determined time interval so as to calculate a blood flow velocity of the target blood vessel. For example, if the contrast agent moved 8 cm from the target blood vessel for the time interval of 0.25 s, then the blood flow velocity of the target blood vessel of the object of measurement may be determined to be 32 cm/s.

The blood flow velocity correction unit 320 may correct the blood flow velocity of the target blood vessel of the object of measurement calculated by the blood flow velocity calculation unit 310. The blood flow velocity correction unit 320 may correct the blood flow velocity calculated by the blood flow velocity calculation unit 310 based on the electrocardiogram data of the object of measurement and the standard blood flow velocity data of the target blood vessel.

The blood flow velocity correction unit 320 may determine a correction factor for correcting the blood flow velocity of the target blood vessel. In order to determine the correction factor, the blood flow velocity correction unit 320 may calculate, from the standard blood flow velocity data, a first average velocity, which is an average value per heart rate cycle of the object of measurement, of the standard blood flow velocity of the target blood vessel, and calculate a second average velocity, which is an average value of the standard blood flow velocity of the target blood vessel between the time when the first medical image is captured and the time when the second medical image is captured. For example, the blood flow velocity correction unit 320 may calculate the first average velocity by Equation 1 below, and calculate the second average velocity by Equation 2 below.

$${v}_{p} = \frac{1}{p 1 - p 0} {\int }_{x = p 0}^{x = p 1} v ( x ) dx \qquad < Equation 1 >$$

where, p1 may refer to an end point in one heart rate cycle, and p0 may refer to a start point in one heart rate cycle. In addition, v(x) may refer to a standard blood flow velocity function.

$${v}_{t} = \frac{1}{t 1 - t 0} {\int }_{x = t 0}^{x = t 1} v ( x ) dx \qquad < Equation 2 >$$

where, t1 may refer to a time point at which the second medical image is captured, and to may refer to a time point at which the first medical image is captured. In addition, v(x) may refer to the standard blood flow velocity function.

The blood flow velocity correction unit 320 may determine the correction factor by using the calculated first average velocity and the calculated second average velocity. For example, the correction factor may be determined by dividing the first average velocity by the second average velocity.

The blood flow velocity correction unit 320 may apply the determined correction factor to the blood flow velocity of the target blood vessel calculated by the blood flow velocity calculation unit 310 so as to correct the blood flow velocity. For example, the blood flow velocity correction unit 320 may multiply the blood flow velocity of the target blood vessel calculated by the blood flow velocity calculation unit 310 by the determined correction factor so as to determine the corrected blood flow velocity. With this configuration, it is possible to obtain data on the blood flow velocity of the target blood vessel of the object of measurement, while excluding or minimizing the influence of the heart rate cycle on blood flow velocity.

The internal configuration of the processor 300 illustrated in FIG. 3 is only an example, and in some examples, configurations other than the illustrated internal configuration may be additionally included, and some configurations may be omitted. For example, if part of the internal configuration is omitted, the processor of the other computing device may be configured to perform the functions of the omitted part of the internal configuration. In addition, although the internal configuration of the processor 300 has been divided by functions and described in FIG. 3, it does not necessarily mean that they are physically separated. In addition, although the blood flow velocity calculation unit 310 and the blood flow velocity correction unit 320 are described herein as separate components from each other for the purpose of facilitating understanding of the disclosure, aspects are not limited thereto.

FIG. 4 is a diagram illustrating a plurality of medical images 420 and 430 for calculating blood flow velocity of a target blood vessel 412.

A first medical image 410 is an image before the contrast agent is injected, and it can be seen that a contrast agent injection unit 414 is connected to the target blood vessel 412 of the object of measurement. In FIG. 4, the target blood vessel 412 in the first medical image 410 is indicated by a dotted line.

It can also be seen that, with the injection of the contrast agent, the contrast agent reaches a first point 422 in the target blood vessel (indicated by a dotted line) in the second medical image 420, and the contrast agent reaches a second point 432 in the target blood vessel in the third medical image 430. In this case, it is considered that the third medical image 430 is captured later than the time when the second medical image 420 is captured.

Based on the second medical image 420 and the third medical image 430, a distance between the first point 422 and the second point 432 may be determined. In addition, a time interval between the time when the second medical image 420 is captured and the time when the third medical image 430 is captured may be determined. For example, the time interval may be determined based on the number of frames between the second medical image 420 and the third medical image 430 and the number of frames per second of a plurality of medical images.

The blood flow velocity of the target blood vessel may be calculated based on the distance between the first point 422 and the second point 432 and the determined time interval. For example, a distance between the first point 422 and the second point 432 may be divided by the determined time interval so as to calculate the blood flow velocity of the target blood vessel.

FIG. 5 is a diagram illustrating a graph 500 including standard blood flow velocity data 510 and electrocardiogram data 520 of the target blood vessel. FIG. 5 illustrates that the electrocardiogram data 520 corresponds to raw data having an r-peak point, but aspects are not limited thereto.

In the graph 500, it can be seen that the standard blood flow velocity data 510 is mapped to the electrocardiogram data 520 such that the cycle of the standard blood flow velocity data 510 of the target blood vessel matches the heart rate cycle of the electrocardiogram data 520. In this case, the standard blood flow velocity data 510 may be a value that is calculated from pre-measured blood flow velocity data of another object of measurement and standardized.

A first time 530 and a second time 540 of the graph 500 represent a start point and an end point in the daily heart rate cycle of the object of measurement, respectively, and a third time 550 and a fourth time 560 represents time points of capturing the medical image for measuring the blood flow velocity of the target blood vessel of the object of measurement, respectively. FIG. 5 illustrates that the third time 550 and the fourth time 560 are positioned between the first time 530 and the second time 540, but aspects are not limited thereto, at least part of the third time 550 and the fourth time 560 may be positioned outside the first time 530 and the second time 540.

The blood flow velocity calculated in FIG. 4 may be corrected based on the standard blood flow velocity data 510 and the electrocardiogram data 520 of the target blood vessel. Specifically, if the correction factor for correcting the blood flow velocity of the target blood vessel is determined, the determined correction factor may be applied to the blood flow velocity calculated in FIG. 4 so as to correct the blood flow velocity. For example, the blood flow velocity calculated in FIG. 4 may be multiplied by the determined correction factor so as to calculate the corrected blood flow velocity.

The correction factor may be determined from the standard blood flow velocity data, based on the first average velocity, which is the average value between the first time 530 and the second time 540 of the standard blood flow velocity of the target blood vessel, and on the second average velocity, which is the average value of the standard blood flow velocity of the target blood vessel between the third time 550 and the fourth time 560. For example, the correction factor may be determined by dividing the first average velocity by the second average velocity. Each of the first average velocity and the second average velocity may be calculated by Equations 1 and 2 described above.

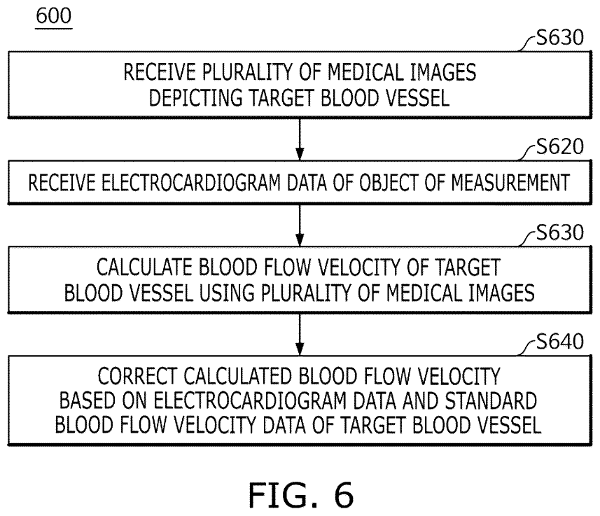

FIG. 6 is a flowchart illustrating part of the process of a method 600 for calculating and correcting blood flow velocity. The method 600 may be initiated by at least one processor (e.g., the processor 220 of the system 200 for correcting blood flow velocity, etc.) receiving a plurality of medical images depicting the target blood vessel of the object of measurement injected with the contrast agent, at S610. The target blood vessel may be one of the right coronary artery (RCA), the left anterior descending artery (LAD), or the left circumflex artery (LCX), but aspects are not limited thereto.

The processor may receive electrocardiogram data of the object of measurement, at S620. The processor may map the standard blood flow velocity data to the electrocardiogram data such that the cycle of the standard blood flow velocity data matches the heart rate cycle of the electrocardiogram data. In this case, the standard blood flow velocity data may be data that is standardized based on at least one of the characteristic information of the object of measurement, such as age, gender, race, height, weight, presence or absence of cardiovascular disease, obesity (BMI), blood pressure, or smoking status of the object of measurement.

The processor may calculate a blood flow velocity of the target blood vessel using a plurality of medical images, at S630. The processor may correct the blood flow velocity calculated based on the electrocardiogram data and the standard blood flow velocity data of the target blood vessel, at S640.

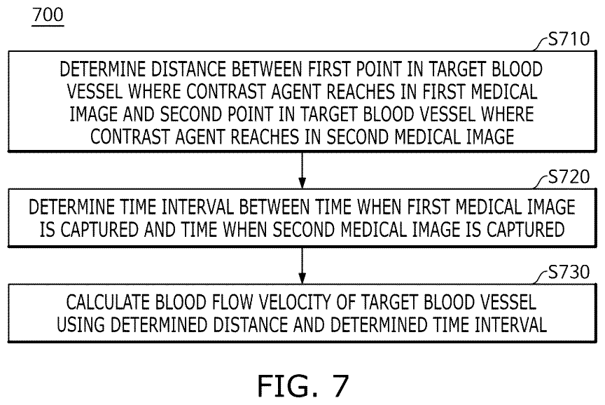

FIG. 7 is a flowchart illustrating a method 700 for calculating blood flow velocity. Each step of the method 700 may correspond to a detailed step of S630 of FIG. 6. A plurality of medical images of FIG. 6 may include a first medical image and a second medical image, and the method 700 may be initiated by the processor determining a distance between a first point in the target blood vessel where the contrast agent reaches in the first medical image and a second point in the target blood vessel where the contrast agent reaches in the second medical image, at S710.

The processor may determine a time interval between time when the first medical image is captured and time when the second medical image is captured, at S720. The time interval may be determined based on, within the plurality of medical images, the number of frames between the first and the second medical images and the number of frames per second of the plurality of medical images.

The processor may calculate a blood flow velocity of the target blood vessel using the determined distance and the determined time interval, at S730.

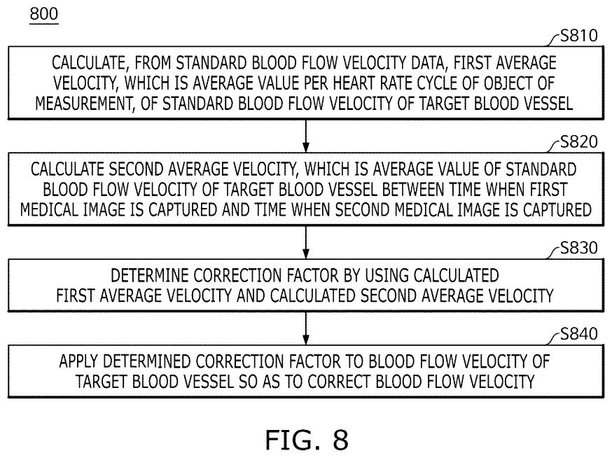

FIG. 8 is a flowchart illustrating a method 800 for correcting blood flow velocity. Each step of the method 800 may correspond to a detailed step of S640 of FIG. 6.

The method 800 may be initiated by the processor calculating, from the standard blood flow velocity data, a first average velocity, which is an average value per heart rate cycle of the object of measurement of the standard blood flow velocity of the target blood vessel, at S810. In this case, the target blood vessel may be one of the right coronary artery (RCA), the left anterior descending artery (LAD), or the left circumflex artery (LCX).

The standard blood flow velocity data may be mapped to the electrocardiogram data such that the cycle of the standard blood flow velocity data matches the heart rate cycle of the electrocardiogram data.

The standard blood flow velocity data may be data that is standardized based on at least one of age, gender, race, height, weight, presence or absence of cardiovascular disease, obesity (BMI), blood pressure, or smoking status of the object of measurement.

The processor may calculate a second average velocity, which is an average value of the standard blood flow velocity of the target blood vessel between the time when the first medical image is captured and the time when the second medical image is captured, at S820.

The processor may determine a correction factor by using the calculated first average velocity and the calculated second average velocity, at S830. The processor may apply the determined correction factor to the blood flow velocity of the target blood vessel so as to correct the blood flow velocity, at S840.

The flowcharts illustrated in FIGS. 6 to 8 and the above description are merely examples, and may be implemented differently in some aspects. For example, one or more operations may be omitted, the order of operations may be changed, one or more operations may be performed in parallel, or one or more operations may be repeatedly performed multiple times.

The method described above may be provided as a computer program stored in a computer-readable recording medium for launch on a computer. The medium may be a type of medium that continuously stores a program executable by a computer, or temporarily stores the program for execution or download. In addition, the medium may be a variety of writing means or storage means having a single piece of hardware or a combination of several pieces of hardware, and is not limited to a medium that is directly connected to any computer system, and accordingly, may be present on a network in a distributed manner. An example of the medium includes a medium configured to store program instructions, including a magnetic medium such as a hard disk, a floppy disk, and a magnetic tape, an optical medium such as a CD-ROM and a DVD, a magnetic-optical medium such as a floptical disk, and a ROM, a RAM, a flash memory, etc. In addition, other examples of the medium may include an app store that distributes applications, a site that supplies or distributes various software, and a recording medium or a storage medium managed by a server.

The methods, operations, or techniques of the present disclosure may be implemented by various means. For example, these techniques may be implemented in hardware, firmware, software, or a combination thereof. Those skilled in the art will further appreciate that various illustrative logical blocks, modules, circuits, and algorithm steps described in connection with the disclosure herein may be implemented in electronic hardware, computer software, or combinations of both. To clearly illustrate this interchangeability of hardware and software, various illustrative components, blocks, modules, circuits, and steps have been described above generally in terms of their functionality. Whether such a function is implemented as hardware or software varies according to design requirements imposed on the particular application and the overall system. Those skilled in the art may implement the described functions in varying ways for each particular application, but such implementation should not be interpreted as causing a departure from the scope of the present disclosure.

In a hardware implementation, processing units used to perform the techniques may be implemented in one or more ASICs, DSPs, digital signal processing devices (DSPDs), programmable logic devices (PLDs), field programmable gate arrays (FPGAs), processors, controllers, microcontrollers, microprocessors, electronic devices, other electronic units designed to perform the functions described in the present disclosure, computer, or a combination thereof.

Accordingly, various example logic blocks, modules, and circuits described in connection with the present disclosure may be implemented or performed with general purpose processors, DSPs, ASICs, FPGAs or other programmable logic devices, discrete gate or transistor logic, discrete hardware components, or any combination of those designed to perform the functions described herein. The general purpose processor may be a microprocessor, but in the alternative, the processor may be any related processor, controller, microcontroller, or state machine. The processor may also be implemented as a combination of computing devices, for example, a DSP and microprocessor, a plurality of microprocessors, one or more microprocessors associated with a DSP core, or any other combination of the configurations.

In the implementation using firmware and/or software, the techniques may be implemented with instructions stored on a computer-readable medium, such as random access memory (RAM), read-only memory (ROM), non-volatile random access memory (NVRAM), programmable read-only memory (PROM), erasable programmable read-only memory (EPROM), electrically erasable PROM (EEPROM), flash memory, compact disc (CD), magnetic or optical data storage devices, etc. The instructions may be executable by one or more processors, and may cause the processor(s) to perform certain aspects of the functions described in the present disclosure.

Although the examples described above have been described as utilizing aspects of the currently disclosed subject matter in one or more standalone computer systems, aspects are not limited thereto, and may be implemented in conjunction with any computing environment, such as a network or distributed computing environment. Furthermore, the aspects of the subject matter in the present disclosure may be implemented in multiple processing chips or apparatus, and storage may be similarly influenced across a plurality of apparatus. Such apparatus may include PCs, network servers, and portable apparatus.

Although the present disclosure has been described in connection with some examples herein, various modifications and changes can be made without departing from the scope of the present disclosure, which can be understood by those skilled in the art to which the present disclosure pertains. In addition, such modifications and changes should be considered within the scope of the claims appended herein.

1. A method for correcting blood flow velocity, the method being performed by one or more processors and comprising:
receiving a plurality of medical images depicting a target blood vessel;
receiving electrocardiogram data of an object of measurement;
determining, using the plurality of medical images, a blood flow velocity of the target blood vessel;
correcting, based on the electrocardiogram data and standard blood flow velocity data of the target blood vessel, the blood flow velocity; and
displaying an indication of the corrected blood flow velocity.

2. The method according to claim 1, wherein the correcting the blood flow velocity comprises:
determining a correction factor for correcting the blood flow velocity of the target blood vessel; and
applying the correction factor to the blood flow velocity of the target blood vessel so as to correct the blood flow velocity.

3. The method according to claim 2, wherein the plurality of medical images comprise a first medical image and a second medical image, and
the determining the correction factor comprises:
determining, from the standard blood flow velocity data, a first average velocity of a standard blood flow velocity of the target blood vessel, wherein the first average velocity is based on an average value per heart rate cycle of the object of measurement;
determining a second average velocity that is based on an average value of a standard blood flow velocity of the target blood vessel between a time associated with the first medical image and a time associated with the second medical image; and
determining, using the first average velocity and the second average velocity, the correction factor.

4. The method according to claim 1, further comprising mapping the standard blood flow velocity data to the electrocardiogram data such that a cycle of the standard blood flow velocity data matches a heart rate cycle of the electrocardiogram data.

5. The method according to claim 1, wherein the plurality of medical images comprise a first medical image and a second medical image, and
the determining the blood flow velocity of the target blood vessel comprises:
determining a distance between a first point in the target blood vessel where a contrast agent reaches in the first medical image and a second point in the target blood vessel where the contrast agent reaches in the second medical image;
determining a time interval between a time associated with the first medical image being captured and a time associated with the second medical image being captured; and
determining, using the determined distance and the determined time interval, the blood flow velocity of the target blood vessel.

6. The method according to claim 5, wherein the time interval is determined based on, within the plurality of medical images, a number of frames between the first medical image and the second medical image, and a number of frames per second.

7. The method according to claim 1, wherein the standard blood flow velocity data is data that is standardized based on at least one of age, gender, race, height, weight, presence or absence of cardiovascular disease, obesity (BMI), blood pressure, or smoking status of the object of measurement.

8. The method according to claim 1, wherein the target blood vessel is one of right coronary artery (RCA), left anterior descending artery (LAD), or left circumflex artery (LCX).

9. A non-transitory computer-readable recording medium storing instructions that, when executed by one or more processors, cause the one or more processors to perform the method according to claim 1.

10. A system comprising:
a communication interface;
a memory; and
one or more processors coupled to the memory and configured to execute one or more computer-readable programs stored in the memory,
wherein the communication interface is configured to:
receive a plurality of medical images depicting a target blood vessel; and
receive electrocardiogram data of an object of measurement, and
wherein the one or more computer-readable programs comprise instructions that, when executed by the one or more processors, cause the system to:
determine, using the plurality of medical images, a blood flow velocity of the target blood vessel;
correct, based on the electrocardiogram data and standard blood flow velocity data of the target blood vessel, the blood flow velocity; and
output an indication of the corrected blood flow velocity.

11. The system according to claim 10, wherein the instructions, when executed by the one or more processors, cause the system to correct the blood flow velocity by:
determining a correction factor for correcting the blood flow velocity of the target blood vessel; and
applying the correction factor to the blood flow velocity of the target blood vessel so as to correct the blood flow velocity.

12. The system according to claim 11, wherein the plurality of medical images comprise a first medical image and a second medical image, and
wherein the instructions, when executed by the one or more processors, cause the system to determine the correction factor by:
determining, from the standard blood flow velocity data, a first average velocity of a standard blood flow velocity of the target blood vessel, wherein the first average velocity is based on an average value per heart rate cycle of the object of measurement;
determining a second average velocity that is based on an average value of a standard blood flow velocity of the target blood vessel between a time associated with the first medical image and a time associated with the second medical image; and
determining, using the first average velocity and the second average velocity, the correction factor.

13. The system according to claim 10, wherein the one or more computer-readable programs further comprise instructions that, when executed by the one or more processors, cause the system to map the standard blood flow velocity data to the electrocardiogram data such that a cycle of the standard blood flow velocity data matches a heart rate cycle of the electrocardiogram data.

14. The system according to claim 10, wherein the plurality of medical images comprise a first medical image and a second medical image, and
wherein the instructions, when executed by the one or more processors, cause the system to determine the blood flow velocity of the target blood vessel by:
determining a distance between a first point in the target blood vessel where a contrast agent reaches in the first medical image and a second point in the target blood vessel where the contrast agent reaches in the second medical image;
determining a time interval between a time associated with the first medical image being captured and a time associated with the second medical image being captured; and
determining, using the determined distance and the determined time interval, the blood flow velocity of the target blood vessel.

15. The system according to claim 14, wherein the time interval is determined based on, within the plurality of medical images, a number of frames between the first medical image and the second medical image, and a number of frames per second.

16. The system according to claim 10, wherein the standard blood flow velocity data is data that is standardized based on at least one of age, gender, race, height, weight, presence or absence of cardiovascular disease, obesity (BMI), blood pressure, or smoking status of the object of measurement.

17. The system according to claim 10, wherein the target blood vessel is one of right coronary artery (RCA), left anterior descending artery (LAD), or left circumflex artery (LCX).
